(19) United States
(12) Patent Application Publication
(10) Pub. No.: US 2021/0220699 A1
(43) Pub. Date: Jul. 22, 2021

(54) Lower limb rehabilitation device

(71) Applicant: Yuhong TAN, Nantong (CN)

(72) Inventors: Yuhong TAN, Nantong (CN); Jingye JI, Nantong (CN); Xiang GU, Nantong (CN)

(21) Appl. No.: 16/771,239

(22) PCT Filed: Dec. 29, 2018
(86) PCT No.: PCT/CN2018/125174

§ 371 Date: Jun. 10, 2020

(30) Foreign Application Priority Data
Dec. 27, 2018 (CN) 201811610520.9

(51) Int. Cl. A63B 23/10; A63B 23/04; C09D 163/00; C09D 7/61; C09D 7/65; C09D 7/80; C09D 7/40

(52) U.S. Cl. CPC A63B 23/10; A63B 23/0405; C09D 163/00; A63B 2209/00; C09D 7/65; C09D 7/80; C09D 7/70; C09D 7/61

(57) ABSTRACT

The invention discloses a lower limb rehabilitation device, which includes a square box body. A square box body is provided on the right side of the square box body. The upper end surface and the right side of the square box body are both hollow surfaces. Block, the upper side of the heightened block is welded with an electric cylinder, the piston rod of the electric cylinder is welded with a first connecting rod, the front and rear side walls of the square box are hinged with connecting plates, and the connecting plates of the front and back The foot fixing plate is directly fixedly connected, and the second connecting rod is welded on the left side of the foot fixing plate.

### TECHNOSPHERE

The invention relates to the technical field of medical equipment, in particular to a lower limb rehabilitation device.

### BACKGROUND TECHNOLOGY

Medical devices refer to instruments, equipment, appliances, in vitro diagnostic reagents and calibrators, materials and other similar or related items used directly or indirectly on the human body, including the required computer software. The utility is mainly obtained through physical methods, not through pharmacological, immunological or metabolic methods, or although these methods are involved, they only play an auxiliary role. The purpose is the diagnosis, prevention, monitoring, treatment or relief of disease; the diagnosis, monitoring, treatment, relief or functional compensation of injury; the examination, substitution, adjustment or support of physiological structure or physiological process; the support or maintenance of life; pregnancy control; Provide information for medical or diagnostic purposes by examining samples from the human body.

For the patients who need to stay in bed for a long time and the lower limbs need to maintain functional positions, the ankle pump exercises are currently used. Currently, there are no instruments that can provide active and passive exercises for the lower limbs to improve patient comfort and compliance. The wear of the equipment is serious, so we need to protect it. To this end, we introduced a lower limb rehabilitation device.

### CONTENTS OF THE INVENTION

The purpose of the present invention is to provide a lower limb rehabilitation device to solve the problems mentioned in the background art.

In order to achieve the above object, the present invention provides the following technical solution: A lower limb rehabilitation device includes a square box body, a square box body is provided on the right side of the square box body, and the upper end surface and the right side of the square box body are hollow Surface, the heightened block is welded on the upper left side of the square box, the electric cylinder is welded on the upper side of the heightened block, the first connecting rod is welded on the piston rod of the electric cylinder, the The front side wall and the rear side wall are hinged with connecting plates, and the two front and rear connecting plates are directly fixedly connected with foot fixing plates, and a second connecting rod is welded on the left side of the foot fixing plates. A steel rope is connected between a connecting rod and a second connecting rod, a limit block is provided on the left side of the foot fixing plate, a first through hole is provided on the connecting plate, and a front side wall of the square box body A second through hole is provided on both the and the rear side wall, a spring rope is connected between the first through hole and the second through hole, and a counter is provided on the front side wall of the square box body.

Preferably, hooks are provided at both ends of the spring rope, a cross section of the limit block is a right triangle, and a counting switch is provided on the limit block.

Preferably, strip-shaped through holes are provided on the front and rear side walls of the square box, and bolts are provided on the square box.

Preferably, a silicone pad is fixedly connected to the upper right side of the square box, and an ankle fixing cloth strap is fixedly connected to the upper side of the silicone pad, and a magic tape is provided on the ankle fixing cloth strap.

Preferably, a fixed elastic band is provided on the upper side of the foot fixing plate, and a heel plastic holder is fixedly connected on the upper side of the foot fixing plate.

Preferably, the number of the first through holes and the second through holes is not less than five.

Preferably, grips are fixed on both sides of the square box, and the grips are provided on an end of the square box away from the foot fixing plate.

Preferably, a non-slip rubber sleeve is fixed on the grip.

Preferably, the foot fixing plate is coated with a wear layer, and the preparation method of the wear layer is:

Take the following raw materials for each component by weight: 11.5-12.8 parts of polyaldehyde resin, 22.3-32.5 parts of epoxy resin, 0.9-1.4 parts of titanate coupling agent, 6.1-6.7 parts of lignin fiber, 11.2 sodium-based bentonite −13.6 parts, propylene glycol methyl ether acetate 1.7-1.9 parts, benzoic acid 0.6-1.4 parts.

S1. Grinding: Mix polyacetal resin, sodium-based bentonite, propylene glycol methyl ether acetate, and benzoic acid and stir them evenly. Perform high-speed grinding in a ball mill until a powder with a mesh number of 35-40 is obtained;

S2. Preparation of color paste: Add epoxy resin, titanate coupling agent, lignin fiber to the powder obtained in S1 and carry out high-speed grinding to a fineness of 28-30 μm to obtain color paste;

S3. Preparation of wear-resistant materials: heating the color paste obtained in S2 to 50-55° C. and holding for 10-30 minutes, and then cooling the temperature to room temperature to obtain wear-resistant materials;

S4. coating: coating the wear-resistant material obtained by S3 to the surface of the foot fixing plate;

S5. shade drying: drying the foot fixing plate obtained by S4 in a cool and ventilated place for 9-10 hours.

Compared with the prior art, the beneficial effect of the present invention is that the present invention is provided with an electric cylinder, which can pull the foot fixing plate for fixing the foot by pulling the electric cylinder, thereby making the patient passive in saving time and effort Receiving rehabilitation treatment, at the same time the patient can actively step on the foot fixing plate for free rehabilitation treatment, the foot fixing plate is coated with a wear-resistant layer, which can reduce wear and prolong the service life Passive and active rehabilitation programs.

### FIGURE ILLUSTRATION

FIG. 1 is a schematic diagram of the structure of the present invention;

2 is a schematic diagram of the top structure of the present invention;

3 is a schematic view of the right side view of the foot fixing plate of the present invention.

In the picture: 1. square box; 2. bolt; 3. spring rope; 4. first through hole; 5. connecting plate; 6. first connecting rod; 7. steel rope; 8. second connecting rod; 9, Electric cylinder; 10, heightening block; 11, square box; 12, counter; 13, limit block; 14, second through hole; 15, strip through hole; 16, foot fixing plate; 17, silicone Cushion; 18, fixed ankle strap; 19, fixed elastic strap; 20, heel plastic support; 21, grip.

### SPECIFIC IMPLEMENTATION MODALITIES

The technical solutions in the embodiments of the present invention will be described clearly and completely in conjunction with the drawings in the embodiments of the present invention. Obviously, the described embodiments are only a part of the embodiments of the present invention, but not all the embodiments. Based on the embodiments of the present invention, all other embodiments obtained by a person of ordinary skill in the art without making creative efforts fall within the protection scope of the present invention.

### Example 1

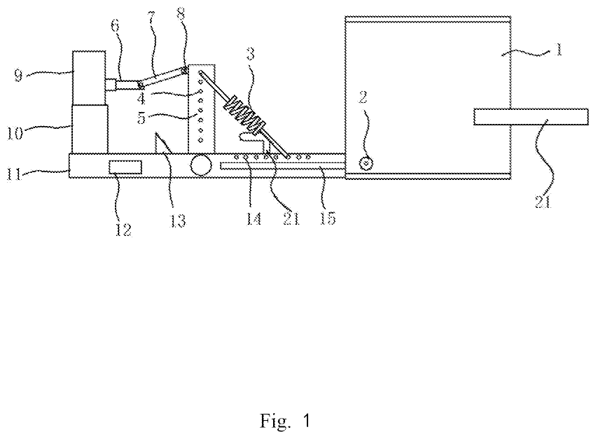

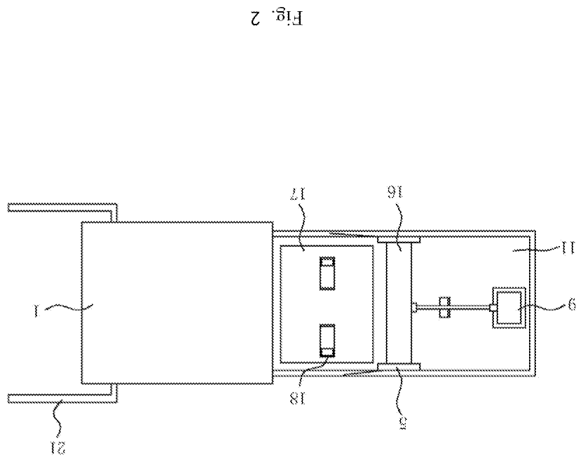

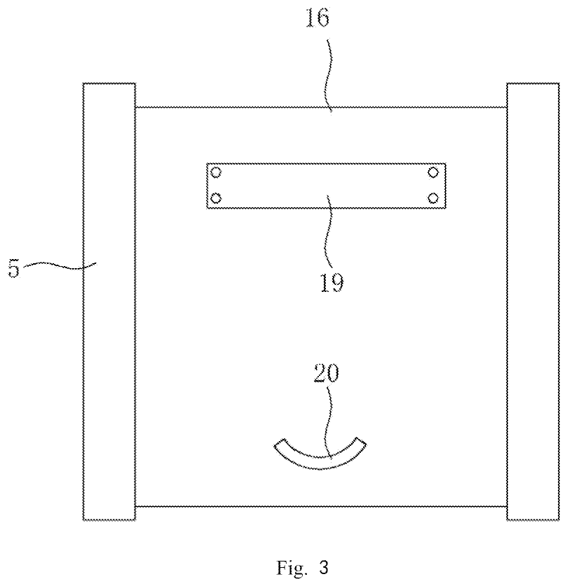

Please refer to FIGS. 1-3, the present invention provides a technical solution: a lower limb rehabilitation device, including a square box 1, a square box 11 is provided on the right side of the square box 1, the square box 11 Both the upper end surface and the right side are hollow surfaces, a heightened block 10 is welded on the upper left side of the square box body 11, an electric cylinder 9 is welded on the upper side of the heightened block 10, and the piston rod of the electric cylinder 9 is welded There is a first connecting rod 6, a connecting plate 5 is hinged on the front and rear side walls of the square box 11, and the two connecting plates 5 in front and back are directly fixedly connected to a foot fixing plate 16, A second connecting rod 8 is welded on the left side of the foot fixing plate 16, a steel rope 7 is connected between the first connecting rod 6 and the second connecting rod 8, and a limit is provided on the left side of the foot fixing plate 16 Block 13, a first through hole 4 is provided on the connecting plate 5, a second through hole 14 is provided on the front and rear side walls of the square box 11, the first through hole 4 and the first A spring rope 3 is connected between the two through holes 14, and a counter 12 is provided on the front side wall of the square box 11.

Specifically, both ends of the spring rope 3 are provided with hooks. The spring rope 3 is connected to the first through hole 4 and the second through hole 14 through the hook. The angle of the plate 16 is fixed to prevent damage to the feet. The limit block 13 is provided with a counting switch, and the counting switch is electrically connected to the counter 12.

Specifically, strip-shaped through holes 15 are provided on the front side wall and the rear side wall of the square box body 11, and bolts 2 are provided on the square box body 1, and the bolts 2 pass through the square box body 1 and the bar shape The through hole 15 connects the square box 1 and the square box 11.

Specifically, a silicone pad 17 is fixedly connected to the upper right side of the square box 11, and an ankle fixing cloth strap 18 is fixedly connected to the upper side of the silicone pad 17. One end is a free end, and the ankle fixing cloth tape 18 is provided with a velcro for fixing a person's ankle.

Specifically, a fixed elastic band 19 is provided on the upper side of the foot fixing plate 16, and a heel plastic holder 20 is fixedly connected on the upper side of the foot fixing plate 16.

Specifically, the number of the first through holes 4 and the second through holes 14 are not less than five.

Specifically, a handle 21 is fixedly arranged on both sides of the square box 1, and the handle 21 is provided at an end of the square box 1 away from the foot fixing plate 16.

Specifically, a non-slip rubber sleeve is fixed on the grip 21.

Working principle: When in use, the legs are extended into the square box 1, the feet are placed on the foot fixing plate 16, the fixed elastic band 19 is elastic, the upper part of the foot is inserted into the fixed elastic band 19 to be fixed, the lower part of the foot On the heel plastic holder 20, the connecting plate 5 is hinged with the square box body 11, so the connecting plate 5 can be driven by the second connecting rod 8, the steel rope 7, the first connecting rod 6, and the electric cylinder 9 to actively drive the foot When you need passive training, you can push the foot fixing plate 16 with your feet to move, and the spring rope 3 can pull the foot fixing plate 16 back, so that you can actively perform repeated exercises. The grip 21 is convenient for the patient Leverage.

### Example 2

The difference from Example 1 is that the foot fixing plate 16 is coated with a wear-resistant layer. The preparation method of the wear-resistant layer is:

Take the following raw materials for each component by weight: 11.5 parts polyacetal resin, 22.3 parts epoxy resin, 0.9 parts titanate coupling agent, 6.1 parts lignin fiber, 11.2 parts sodium bentonite, 1.7 parts propylene glycol methyl ether acetate Parts, 0.6 parts of benzoic acid.

S1. Grinding: Mix polyacetal resin, sodium-based bentonite, propylene glycol methyl ether acetate, and benzoic acid and stir them evenly. Perform high-speed grinding in a ball mill until a powder with a mesh number of 40 is obtained;

S2. Preparation of color paste: adding epoxy resin, titanate coupling agent, lignin fiber to the powder obtained in S1 and grinding at high speed to a fineness of 28 μm to obtain a color paste;

S3. Preparation of wear-resistant materials: heating the color paste obtained in S2 to 55° C. and holding for 10 minutes, and then cooling the temperature to room temperature to obtain wear-resistant materials;

S4, coating: coating the wear-resistant material obtained by S3 to the surface of the foot fixing plate 16;

S5, shade drying: drying the foot fixing plate 16 obtained in S4 in a cool and ventilated place for 10 hours.

In this embodiment, the patient achieves the purpose of exercise through the foot fixing plate 16. The foot fixing plate 16 slides back and forth on the square box 11, which will cause greater wear, so the foot fixing plate 16 is coated with a layer of wear The layer can alleviate the wear caused when the foot fixing plate 16 slides, protect the foot fixing plate 16, and greatly extend its service life.

### Example 3

The difference from Example 2 is that the specific gravity of the formula is modified. The modified formula is:

Take the following raw materials for each component by weight: 12.8 parts of polyaldehyde resin, 32.5 parts of epoxy resin, 1.4 parts of titanate coupling agent, 6.7 parts of lignin fiber, 13.6 parts of sodium bentonite, 11.9 parts of propylene glycol methyl ether acetate Parts, benzoic acid 1.4 parts.

S1. Grinding: Mix polyacetal resin, sodium-based bentonite, propylene glycol methyl ether acetate, and benzoic acid and stir them evenly. Perform high-speed grinding in a ball mill until a powder with a mesh number of 40 is obtained;

S2. Preparation of color paste: adding epoxy resin, titanate coupling agent, lignin fiber to the powder obtained in S1 and grinding at high speed to a fineness of 28 μm to obtain a color paste;

S3. Preparation of wear-resistant materials: heating the color paste obtained in S2 to 55° C. and holding for 10 minutes, and then cooling the temperature to room temperature to obtain wear-resistant materials;

S4. Coating: coating the wear-resistant material obtained by S3 to the surface of the foot fixing plate 16;

S5. Shade drying: drying the foot fixing plate 16 obtained in S4 in a cool and ventilated place for 10 hours.

In this embodiment, the proportion of the formula is modified and kept for comparison, so as to obtain the optimal formula.

### Example 4

The difference from Example 2 is that the specific gravity of the formula is modified. The modified formula is:

Take the following raw materials for each component by weight: 12.2 parts of polyaldehyde resin, 27.4 parts of epoxy resin, 1.2 parts of titanate coupling agent, 6.4 parts of lignin fiber, 12.4 parts of sodium bentonite, and propylene glycol methyl ether acetate 1.8 Parts, 1 part benzoic acid.

S1. Grinding: Mix polyacetal resin, sodium-based bentonite, propylene glycol methyl ether acetate, and benzoic acid and stir them evenly. Perform high-speed grinding in a ball mill until a powder with a mesh number of 40 is obtained;

S2. Preparation of color paste: adding epoxy resin, titanate coupling agent, lignin fiber to the powder obtained in S1 and grinding at high speed to a fineness of 28 μm to obtain a color paste;

S3. Preparation of wear-resistant materials: heating the color paste obtained in S2 to 55° C. and holding for 10 minutes, and then cooling the temperature to room temperature to obtain wear-resistant materials;

S4. coating: coating the wear-resistant material obtained by S3 to the surface of the foot fixing plate 16;

S5. shade drying: drying the foot fixing plate 16 obtained in S4 in a cool and ventilated place for 10 hours.

In this embodiment, the proportion of the formula is modified and kept for comparison, so as to obtain the optimal formula.

The abrasion resistance and peel strength of the foot fixing plate 16 coated with abrasion-resistant paints with different specific gravity in Examples 1, 2, 3, and 4 were tested. The data is normalized.

TABLE 1

| | Wear resistance | Peel strength |
|---|---|---|
| Example 1 | 100% | 100% |
| Example2 | 176% | 134% |
| Example3 | 179% | 136% |
| Example4 | 184% | 141% |

The patient achieves the purpose of exercise through the foot fixing plate 16. The foot fixing plate 16 slides back and forth on the square box 11, which will cause greater wear and tear. Therefore, a wear-resistant layer is coated on the foot fixing plate 16 to relieve the feet The wear caused by the sliding portion of the fixing plate 16 protects the fixing plate 16 of the foot, which greatly prolongs its service life. As can be seen from the data in the table, after coating the wear-resistant material, the wear resistance of the foot fixing plate 16 has been significantly enhanced, and Example 4 has the best effect, so the optimal raw material formula determined is: polyaldehyde Resin 12.2 parts, epoxy resin 27.4 parts, titanate coupling agent 1.2 parts, lignin fiber 6.4 parts, sodium bentonite 12.4 parts, propylene glycol methyl ether acetate 1.8 parts, benzoic acid 1 part.

Although the embodiments of the present invention have been shown and described, those of ordinary ski II in the art can understand that various changes, modifications, and substitutions can be made to these embodiments without departing from the principles and spirit of the present invention And variations, the scope of the invention is defined by the appended claims and their equivalents.

1. A lower limb rehabilitation device, comprising a square box (1), characterized in that: a square box (11) is provided on the right side of the square box (1), and the upper side of the square box (11) Both the end surface and the right side are hollow surfaces, and a heightening block (10) is welded on the upper left side of the square box (11), and an electric cylinder (9) is welded on the upper side of the heightening block (10). The piston rod of the cylinder (9) is welded with a first connecting rod (6), and a connecting plate (5) is hinged on the front and rear side walls of the square box (11). The connecting plate (5) is directly fixedly connected with a foot fixing plate (16), and a second connecting rod (8), the first connecting rod (6) and the first connecting rod are welded on the left side of the foot fixing plate (16) A steel rope (7) is connected between the two connecting rods (8), a limit block (13) is provided on the left side of the foot fixing plate (16), and a first through hole is provided on the connecting plate (5) (4) The front and rear side walls of the square box (11) are provided with second through holes (14), one of the first through holes (4) and the second through holes (14) A spring rope (3) is connected between them, and a counter (12) is provided on the front side wall of the square box (11).

2. A lower limb rehabilitation device according to claim 1, characterized in that: both ends of the spring rope (3) are provided with hooks, and the cross section of the limiting block (13) is a right triangle There is a counting switch on the limit block (13).

3. A lower limb rehabilitation device according to claim 1, characterized in that strip-shaped through holes (15) are provided on the front and rear side walls of the square box (11), the square The box (1) is provided with bolts (2).

4. The lower limb rehabilitation device according to claim 1, characterized in that: a silicone pad (17) is fixedly connected to the upper right side of the square box (11), and a silicone pad (17) is fixedly connected to the upper side of the silicone pad (17) An ankle fixing cloth strap (18), the ankle fixing cloth strap (18) is provided with a velcro.

5. The lower limb rehabilitation device according to claim 1, characterized in that: a fixed elastic band (19) is provided on the upper side of the foot fixing plate (16), and an upper side of the foot fixing plate (16) The fixed connection has a heel plastic bracket (20).

6. The lower limb rehabilitation device according to claim 1, wherein the number of the first through holes (4) and the second through holes (14) are not less than five.

7. The lower limb rehabilitation device according to claim 1, characterized in that: a handle (21) is fixedly arranged on both sides of the square box (1), and the handle (21) is provided on the square box (1) One end away from the foot fixing plate (16).

8. A lower limb rehabilitation device according to claim 7, characterized in that the grip (21) is fixedly provided with a non-slip rubber sleeve.

9. The lower limb rehabilitation device according to claim 1, characterized in that: the foot fixing plate (16) is coated with a wear-resistant layer, and the formula of the wear-resistant layer is:
Take the following raw materials for each component by weight: 11.5-12.8 parts of polyaldehyde resin, 22.3-32.5 parts of epoxy resin, 0.9-1.4 parts of titanate coupling agent, 6.1-6.7 parts of lignin fiber, 11.2 sodium-based bentonite −13.6 parts, propylene glycol methyl ether acetate 1.7-1.9 parts, benzoic acid 0.6-1.4 parts.

10. A lower limb rehabilitation device according to claim 9, wherein the manufacturing method of the wear-resistant layer is:
S1. Grinding: Mix polyacetal resin, sodium-based bentonite, propylene glycol methyl ether acetate, and benzoic acid and stir them evenly. Perform high-speed grinding in a ball mill until a powder with a mesh number of 35-40 is obtained;
S2. Preparation of color paste: Add epoxy resin, titanate coupling agent, lignin fiber to the powder obtained in S1 and carry out high-speed grinding to a fineness of 28-30 μm to obtain color paste;
S3. Preparation of wear-resistant materials: heating the color paste obtained in S2 to 50-55° C. and holding for 10-30 minutes, and then cooling the temperature to room temperature to obtain wear-resistant materials;
S4. coating: coating the wear-resistant material obtained by S3 to the surface of the foot fixing plate (16);
S5. shade drying: Dry the foot fixing plate (16) obtained in S4 in a cool and ventilated place for 9-10 hours.
